(19) United States
(12) Patent Application Publication
(10) Pub. No.: US 2014/0142181 A1
(43) Pub. Date: May 22, 2014

(54) TREATING AND INHIBITING RECURRENCE OF MUSCLE CRAMPS

(71) Applicant: Stanley L. Mills, Goldsby, OK (US)

(72) Inventor: Stanley L. Mills, Goldsby, OK (US)

(21) Appl. No.: 13/681,006

(22) Filed: Nov. 19, 2012

(51) Int. Cl. A61K 31/197

(52) U.S. Cl. CPC A61K 31/197
USPC 514/565

(57) ABSTRACT

A method of topical application of a creatine composition for treating and/or inhibiting recurrence of muscle cramps/spasms as well as muscle pain and/or stiffness associated therewith.

### BACKGROUND

The presently disclosed and claimed inventive concept(s) relates to methods of using creatine compositions for the treatment and/or inhibition of recurrence of muscle cramps, muscle stiffness, muscle pain, and muscle spasms.

Although a multitude of events exist which can be described as muscle cramps, such as: heat cramps, writer's cramp, and nocturnal or night cramps, to elucidate a few, one common thread emerges: all demonstrate an involuntary and forcible muscle contraction that does not subside for a period of time. The contracted muscle may persist for seconds or in some cases minutes or longer. Moreover, it is not uncommon for these events to reoccur immediately after the contraction subsides and/or after resuming the activity or exercise that prompted the initial incident. It is the rare athlete who experiences a muscle cramp that can quickly resume the competition, activity, or conditioning exercise unimpeded. The more typical experience is the inability of the individual to continue and compete at what is a normal level of output for that individual. Nocturnal cramps frequently persist and reoccur multiple times per night, or night after night, even with massaging and stretching of the muscle to alleviate the initial cramp. Medical and popular writers have proposed numerous physiological causes for over 100 years yet to date, effective treatments and preventive measures remain elusive, as does an understanding of the causative agent(s) for muscular cramps.

Products like GATORADE™ appear to be effective at reducing the incidence of heat related injuries including heat syncope, heat exhaustion, dehydration syndrome, and heat stroke by increasing the available electrolytes, fluid, and energy sources. However, these “sports drinks” have failed to decrease the incidence of muscle cramps over multiple decades of use in amateur or professional athletes. Consider the common practice of intravenous administration of fluids and electrolytes in the collegiate and professional athletes. Intravenous administration both before the competitive event and following the muscle cramp during the event has demonstrated limited utility of this practice for prevention of initial and reoccurring muscular cramps.

Quinine has been used in treatment for muscle cramps, however, on Dec. 11, 2006 the U.S. Food and Drug Administration (USFDA) published a cease and discontinue manufacturing order for all products containing quinine except one approved for use as an antimalarial. The USFDA further cautioned consumers about off-label use of quinine to treat leg cramps. The USFDA new release indicated that the agency received 665 reports of adverse events with serious outcomes associated with quinine use, including 93 deaths. Quinine drugs are associated with serious side effects, such as cardiac arrhythmias, thrombocytopenia (a decrease in blood platelets that can cause hemorrhage or clotting problems), and severe hypersensitivity reactions. There is also the potential for serious interactions between quinine drugs and other drugs, and there are conditions under which quinine should not be used.

Creatine is generated endogenously predominantly by the liver, kidneys, and a lesser amount by the pancreas at a rate of about one g/d. Omnivorous diets add an additional one g/d. The human body stores approximately 95% of produced and consumed creatine in skeletal muscle. Creatine is possibly the most researched of all natural supplements. Researchers are both focused on its potential as an ergogenic aid for improving performance and health in athletes, and its impact on various pathological conditions (Cooper, R., et al. [2012] “Creatine supplementation with specific view to exercise/sports performance: an update,” in J. Int. Soc. Sports Nutr. 9:33). However, there has been a widespread belief that there is an increased incidence of cramping associated with creatine supplementation during training and competition. In one study, conducted to elucidate increased cramping as a potential side effect of creatine use in athletes, 39 Division I baseball players were evaluated in a double blind study. The researchers reported no significant difference between the creatine group and non-creatine group regarding cramping. (Greenwood, M., et al., [2003] “The effects of creatine supplementation on cramping and injury occurrence during college baseball training and competition,” JEPonline 6(4): 16-23).

About one in every three adults are affected by lower limb muscle cramps and the number increases to one in every two adults over the age of 60 years. Most patients experience little or no relief with current therapies used to prevent muscle cramps (Blyton, F., Chuter, V., and Burns, J. [2012] “Unknotting night-time muscle cramp: a survey of patients experience, help-seeking behavior and perceived treatment effectiveness,” Journal of Foot and Ankle Research. 5:7). Most of the therapies used to treat muscle cramps (such as orally-administered ibuprofen or muscle relaxants) are directed to reducing the associated symptoms such as pain, and do not quickly alleviate the cramp, and do not prevent its reoccurrence with the resumption of activity. It is to such a quick-acting therapy which also reduces the short-term recurrence of cramping that the presently disclosed and claimed inventive concept(s) is directed.

### SUMMARY

The presently disclosed and claimed inventive concept(s) relates to methods of topical administration of a creatine composition for the treatment of muscle cramps and for the short term, (e.g., for up to 24 hours) prevention of recurrence of muscle cramps, stiffness, pain and spasms. The creatine composition may also comprise an effective amount of at least one essential oil in a topical preparation. The muscle cramps, muscle spasms, muscle pain, and/or muscle stiffness may be exercise-associated, nocturnal, premenstrual, or age-related, for example.

### DETAILED DESCRIPTION OF THE INVENTIVE CONCEPT(S)

The presently disclosed and claimed inventive concept(s) relates to methods of using creatine compositions to both treat, and inhibit the short term recurrence of muscle cramps, muscle pain, muscle spasms, and muscle stiffness. The creatine composition may be topically administered alone with a carrier, or in combination with an effective amount of at least one essential oil. More particularly, the presently disclosed and claimed inventive concept(s) also contemplates the treatment and prevention of short term recurrence of exercise-associated muscle cramps, pain, stiffness and spasms, utilizing an effective amount of at least one creatine compound in combination with an effective amount of the at least one essential oil. Administration of the composition to the area of the body which is experiencing discomfort generally results in alleviation of the cramping and pain within 3-10 minutes and generally within at least 20 minutes.

Further, administration of the creatine composition to the area of the body (e.g., leg, arm, back, foot) which is experiencing cramping generally prevents cramping from returning (recurring) within a period of 2-6 hours and up to 12 hours, 18 hours, or 24 hours after the initial episode of muscle cramping, pain, stiffness or spasm.

In one embodiment of the presently disclosed and claimed inventive concept(s), for example, the composition contains creatine monohydrate, chamomile oil, clary sage oil, lavender oil, and eucalyptus oil in a lotion.

Before explaining at least one embodiment of the presently disclosed and claimed inventive concept(s) in more detail by way of exemplary description, examples, and results, it is to be understood that the presently disclosed and claimed inventive concept(s) is not limited in its application to the details of methods and compositions as set forth in the following description. The presently disclosed and claimed inventive concept(s) is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently claimed and disclosed inventive concept(s) may be practiced without these specific details. In other instances, features which are well known to persons of ordinary skill in the art have not been described in detail to avoid unnecessary complication of the description.

Unless otherwise defined herein, scientific and technical terms used in connection with the presently disclosed and claimed inventive concept(s) shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and methods of their application disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined herein.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” Throughout this application, the term “about” is used to indicate that a value includes the inherent variation of error for the composition, the method used to administer the composition, or the variation that exists among the study subjects. The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The term “naturally-occurring” as used herein as applied to an object refers to the fact that an object can be found in nature. For example, an essential oil that is present in a source that can be isolated from a source in nature and which has not been intentionally modified by man in the laboratory or otherwise, other than to be purified, is naturally-occurring.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” or “substantially pure” means an object species (e.g., a particular essential oil) is the predominant species present (i.e., on a molar basis it is more abundant than any other essential oil in the composition thereof), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species. The term “pure” or “substantially pure” also refers to preparations where the object species (e.g., the essential oil) is at least 60% (w/w) pure, or at least 70% (w/w) pure; or at least 75% (w/w) pure; or at least 80% (w/w) pure; or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “subject” as used herein refers to mammals. “Mammal” for purposes of treatment herein refers to any animal classified as a mammal, including human, nonhuman primates, and monkeys, domestic and farm animals including horses, and other equines, dogs, cats, and any other animal that has mammary tissue which suffers from muscle cramps, pain, stiffness or spasms.

“Treatment” refers to therapeutic treatments. “Prevention” refers to prophylactic or preventative treatment measures. “Inhibition of recurrence” refers to a treatment which is applied to a subject who recently had a muscle cramp or spasm for prevention of a recurrence within a period of up to 24 hours. Those in need of treatment include, but are not limited to, individuals already having muscle cramps, spasms, pain, or stiffness as well as individuals who are at risk of having a recurrence of such condition (e.g., those who recently suffered such a condition, for example because of vigorous physical activity). The term “treating” refers to administering the composition to a patient for therapeutic purposes to eliminate or reduce the effect or inhibit recurrence.

A “therapeutic composition” or “pharmaceutical composition” refers to a creatine-containing composition that may be administered topically to bring about a therapeutic effect or to inhibit or reduce the recurrence of the muscle cramp, spasm, pain, or stiffness.

“Topical”, when used to define a mode of administration, means that a material is administered by being applied to the skin.

The term “effective amount” refers to an amount of a creatine compound and/or essential oil which is sufficient to exhibit a detectable therapeutic effect without undue adverse side effects (such as toxicity, irritation and allergic response) commensurate with a reasonable benefit/risk ratio when used in the manner of the inventive concept(s). The therapeutic effect may include, for example but not by way of limitation, a partial or complete relaxation of a muscle cramp, muscle spasm, muscle pain, or muscle stiffness. The effective amount for a particular subject will depend upon the type of subject, the subject's size and health, the nature and severity of the condition, muscle cramp, muscle spasm, muscle pain or muscle stiffness, to be treated, the quickness of administration, the specific formulations employed, and the like. Thus, it is not possible to specify an exact effective amount in advance. However, the effective amount for a given situation can be determined by one of ordinary skill in the art, for example, an athletic trainer or physician, experience using routine experimentation based on the information provided herein, for example by providing the subject with one or more doses of the creatine composition. For example, the effective unit dose may comprise from about 0.1 g to about 3 g of the creatine compound.

As used herein, the term “concurrent therapy” is used interchangeably with the terms “combination therapy” and “adjunct therapy”, and will be understood to mean that the subject in need of treatment is treated or given another drug for the condition (e.g., ibuprofen or a muscle relaxant) in conjunction with the pharmaceutical compositions of the presently disclosed and claimed inventive concept(s). This concurrent therapy can be sequential therapy, where the patient is treated first with one composition and then the other, or the two compositions are given simultaneously.

The terms “administration” and “administering”, as used herein will be understood to include topical administration which may be understood to include transdermal passage of the active agent. In addition, the compositions of the presently disclosed and claimed inventive concept(s) (and/or the methods of administration of same) may be designed to provide delayed, controlled or sustained release using formulation techniques which are well known in the art, wherein for example, a creatine compound continues to be released to the skin for some minutes or hours after initial topical application.

The presently disclosed and claimed inventive concept(s) includes a therapeutic composition comprising a therapeutically-effective or pharmaceutically-effective amount of at least one active ingredient described herein in combination with a pharmaceutically acceptable carrier. As used herein, a “pharmaceutically acceptable carrier” is a pharmaceutically acceptable solvent, suspending agent or vehicle for delivering the compositions of the presently disclosed and claimed inventive concept(s) to the subject. The carrier may be liquid or solid and is selected with the planned manner of administration in mind. Examples of pharmaceutically acceptable carriers that may be utilized in accordance with the presently disclosed and claimed inventive concept(s) include, but are not limited to, polyethylene glycol (PEG), polymers, liposomes, ethanol, DMSO, aqueous buffers, solvents, oils, DPPC, lipids, and combinations thereof.

More specifically, “pharmaceutically-acceptable topical carrier” and equivalent terms may refer to an inactive liquid or cream vehicle capable of suspending or dissolving the creatine compound and optionally an essential oil compound described or referred to herein and having the properties of being generally nontoxic and noninflammatory when applied to the skin. This term is specifically intended to encompass carrier materials approved for use in topical cosmetics and topical and systemic pharmaceuticals. Representative carriers include water, silicone fluids, oils, both vegetable and mineral, cream bases, lotion bases, ointment bases and the like. These bases may include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21^{st} ed., 2005, which is hereby expressly incorporated herein by reference in its entirety.

One non-limiting embodiment of a lotion which may be used in the composition of the presently disclosed and claimed inventive concept(s) is shown in Table 1.

TABLE 1
Lotion Example

| Phase | Material | % Nominal |
|---|---|---|
| A | Aqua | 75.00 |
| | Disodium EDTA | 0.1 |
| | Glycereth-26 | 2.0 |
| B | Myristyl Myristate | 1.0 |
| | Cetearyl Alcohol and | 5.0 |
| | Glyceryl Stearate and | |
| | PEG-40 Stearate and | |
| | Ceteareth-20 | |
| | Cetearyl Alcohol | 2.0 |
| | Neopentyl Glycol Dicaprylate/Dicaprate | 2.0 |
| | Cetyl Esters | 1.0 |
| | PPG-26 Oleate | 3.0 |
| | Chamomile Oil | 0.53 |
| | Eucalyptus Oil | 0.30 |
| | Clary Sage Oil | 0.15 |
| | Lavender Oil | 0.53 |
| | N-Guanyl-N-methylglycine | 7.00 |
| | Phenoxyethanol and | 0.5 |
| | Methylparaben and | |
| | Propylparaben and | |
| | Butylparaben | |

One non-limiting embodiment of a cream which may be used in the composition of the presently disclosed and claimed inventive concept(s) is shown in Table 2.

TABLE 2
Cream Example

| Phase | Material | % Nominal |
|---|---|---|
| A | Aqua | 76.89 |
| | Magnesium Aluminum Silicate | 0.75 |
| | Disodium EDTA | 0.10 |
| | Xanthan Gum | 0.25 |
| | Glycerin | 4.00 |
| | Sucrose Stearate | 1.50 |
| B | Glyceryl Cocoate | 1.00 |
| | Polyglyceryl-3 Oleate | 3.00 |
| | Cetearyl Alcohol | 1.50 |
| | Cetyl Esters | 2.00 |
| | Chamomile Oil | 0.53 |
| | Eucalyptus Oil | 0.30 |
| | Clary Sage Oil | 0.15 |
| | Lavender Oil | 0.53 |
| | N-Guanyl-N-methylglycine | 7.00 |
| | Phenoxyethanol and | 0.50 |
| | Methylparaben and | |
| | Propylparaben and | |
| | Butylparaben | |

One non-limiting embodiment of an ointment which may be used in the composition of the presently disclosed and claimed inventive concept(s) is shown in Table 3.

TABLE 3
Ointment Example

| Material | % Nominal |
|---|---|
| Cholesterol | 3.00 |
| Cottonseed oil | 1.47 |
| Chamomile Oil | 0.54 |
| Eucalyptus Oil | 0.30 |
| Clary Sage Oil | 0.15 |
| Lavender Oil | 0.54 |
| N-Guanyl-N-methylglycine | 7.10 |
| White Petrolatum | 86.90 |

One non-limiting embodiment of a gel which may be used in the composition of the presently disclosed and claimed inventive concept(s) is shown in Table 4.

TABLE 4
Gel Example

| Phase | Material | % Nominal |
|---|---|---|
| A | Chamomile Oil | 0.53 |
| | Eucalyptus Oil | 0.30 |
| | Clary Sage Oil | 0.15 |
| | Lavender Oil | 0.53 |
| | Jojoba Oil | 28.49 |
| | Span 60 | 1.08 |
| | Hydroxypropyl methylcellulose (HPMC) | 1.00 |
| | Carbopol 934 P | 0.20 |
| | N-Guanyl-N-methylglycine | 7.10 |
| B | Aqua | 53.70 |
| | Brij 35 | 1.92 |
| | Propylene glycol | 5.00 |
| C | Triethanolamine (TEA) | Q.S. |

The terms “liposome”, “lipid nanostructure” and “vesicle” may be used interchangeably herein and will be understood to refer to an assembled structure constructed of molecules such as lipids and/or proteins, for example, not through covalent bonds but through interactions (such as but not limited to, hydrophobic interactions, electrostatic interactions and hydrogen bonds) acting between the molecules in an aqueous medium.

The terms “aqueous solution” and “aqueous medium” may be used interchangeably herein and will be understood to refer to water as well as any kind of solution which is physiologically acceptable and solvent in water.

The creatine compositions of the presently disclosed and claimed inventive concept(s) may be prepared according to methods known in the art, particularly in light of the disclosure and examples set forth herein. The starting materials used to synthesize the creatine compositions of the presently disclosed and claimed inventive concept(s) are commercially available or capable of preparation using methods known in the art.

The presently disclosed and claimed inventive concept(s) is also directed to a method of using any of the pharmaceutical compositions described herein above. Said method includes the steps of providing the pharmaceutical composition and administering an effective amount of the pharmaceutical composition to a subject in need thereof.

Where used herein the term “emulsifier” refers to a compound which is used to promote and maintain a stable mixture or dispersion (emulsion) of oil droplets in a water phase, or water droplets in an oil phase. Emulsifiers are, essentially, surfactants. These surfactants can be ionic (cationic or anionic) or non-ionic, and they can be used alone or in combination. Emulsifiers contemplated for use herein include, but are not limited to, cetearyl alcohol and sodium cetearyl sulfate, PEG-1000 monocetyl ether, glycol stearate, glyceryl stearate, cetyl alcohol, PEG-100 stearate, ceteareth-20, or quaternary ammonium salts such as alkyl trimethyl ammonium bromide, the polyol ester glycerol monostearate, potassium stearate, sodium lauryl sulfate, and ethoxylated fatty alcohols. Fatty acids like stearic acids may be included to regulate the consistency of the emulsion. Finally, polymers such as carbomers can be included in small amounts to stabilize the emulsion.

“Penetration enhancers” are substances which enhance passage of topically-applied compounds into the stratum corneum of the skin and therefrom into the epidermis and dermis. Examples include, but are not limited to: dimethylisosorbide, ethoxydiglycol, 1-dodecylazacycloheptan-2-one, propylene glycol, oleyl alcohol, polyoxyethylene ester, sorbitan mono-9-octadecenoate, poly(oxy-1,2-ethanediyl) and derivatives thereof, ethanol, glyceryl monoethyl ether, monoglycerides, isopropylmyristate, lauryl alcohol, lauric acid, lauryl lactate, terpinol, menthol, D-limonene, beta-cyclodextrin, DMSO (dimethyl sulfoxide), polysorbates, fatty acids (e.g., oleic), bile salts, N-methylpyrrolidone, polyglycosylated glycerides, 1-dodecylazacycloheptan-2-one (Azone®), Cyclopentadecalactone (CPE-215®), Alkyl-2-(N,N-disubstituted amino)-alkanoate ester (NexAct®), 2-(n-nonyl)-1,3-dioxolane (DEPA®), and penetration enhancers shown for example in U.S. Pat. Nos. 3,909,816; 4,405,616; 4,801,586; 4,861,764; 4,886,783; 4,983,396; 5,118,845; and 5,196,410, each of which is hereby expressly incorporated herein by reference in its entirety.

The term “creatine” or “creatine compound” as used herein refers to any of the numerous forms of commercially-available creatine which can be used in the composition of the presently disclosed and claimed inventive concept(s) as well as any new forms to be developed in the future which comprise a bio-active form of creatine. Examples of such forms of creatine compounds include, but are not limited to, creatine monohydrate, creatine anhydrous, creatine HCl, and esters such as creatine pyruvate, creatine citrate, creatine malate, creatine phosphate, magnesium creatine, creatine oroate, Kre Alkalyn, creatine ethyl ester, creatine ethylester (HCL), and creatine gluconate.

“Essential oil” or “essential oils,” as used herein, refers to synthetic or natural oils derived from any source, but particularly from the flowers, seeds, fruits, roots, bark, sap, herbs, trees, and other plants. Such essential oils can generally be extracted by several methods known to those of skill in the art. When these types of oils are exposed to air they tend to evaporate (i.e., a volatile oil). Essential oils are insoluble in water and are soluble in alcohol, ether, fixed oils (vegetal), and other organic solvents. Essential oils are generally aromatic and are typically are named for the plant from which the oil is extracted. For example, rose oil or peppermint oil are derived from rose or peppermint plants, respectively. Examples of essential oils that can be used in the context of the presently disclosed and claimed inventive concept(s) include, but are not limited to, sesame oil, macadamia nut oil, tea tree oil, evening primrose oil, Spanish sage oil, Spanish rosemary oil, coriander oil, thyme oil, pimento berries oil, rose oil, almond oil, anise oil, balsam oil, bergamot oil, rosewood oil, camphor oil, cardoman oil, cedar oil, cedar leaf oil, chamomile oil, cinnamon oil, sage oil, clary sage oil, clove oil, clove leaf oil, cypress oil, eucalyptus oil, fennel oil, fistree oil, sea fennel oil, frankincense oil, geranium oil, ginger oil, grapefruit oil, jasmine oil, jojoba oil, juniper oil, lavender oil, lemon oil, lemongrass oil, lime oil, mandarin oil, marjoram oil, myrrh oil, menthol, neroli oil, orange oil, patchouli oil, pepper oil, black pepper oil, petitgrain oil, pine seed oil, pine needle oil, rose otto oil, rosemary oil, sandalwood oil, spearmint oil, spikenard oil, vetiver oil, walnut oil, whitepine oil, wintergreen oil, or ylang ylang. Other aromatic essential oils known to those of skill in the art which are not listed here are also contemplated as being useful within the context of the present invention.

“Pharmaceutically acceptable topical preparation” as used herein, refers to gels, creams, ointments, pastes, lotions, sprays or suspensions intended for external application to the body of a mammal such as, but not limited to, a human or other primate, a horse, a dog, or a cat.

The term “exercise-associated muscle cramps” as used herein, refers to a spontaneous, painful and frequently prolonged contraction of a single or a multiple muscle groups as a result of strenuous exercise. The muscle cramp may appear without warning or may be preceded by a “spasm” which refers herein to a transient abnormal contraction of single or multiple muscle groups. If an athlete continues to engage in the activity that prompted the spasm, a cramp typically ensues. Cramping or muscle spasm is a common complaint in athletes and has multiple causes. There is a distinction between the cramp that occurs with effort or injury and the cramp that occurs at rest at night (nocturnal cramp). Without wishing to be bound by theory, it is though that cramps in athletes may occur during exercise when a relative arterial insufficiency in blood flow arises or when transient ischemia caused by isometric contractions in overused muscle groups occurs. Injury to the muscle, by a blow that causes slight infiltration of blood, overstretching of a muscle with resultant tearing of some fibers, or strain by overcontraction of the muscle against resistance frequently leads to muscle cramps or spasm. Some cramps may appear without warning and without any apparent etiology. In a hot climate, overheating, or excessive sweating leading to a decrease in body electrolyte content, may cause muscle spasm. Sudden chilling may cause cramping or spasm. A build-up of carbon dioxide or lactic acid in the muscle and a decrease in oxygenation may also cause cramping.

The term “nocturnal cramps” as used herein, refers to spontaneous, painful and frequently prolonged contraction of a single or multiple muscle groups that occur typically at night and in a resting state. The etiology of nocturnal cramps is unknown and remains elusive. It occurs in all age groups including infants, adolescents, adults, and the elderly. It appears to affect males and females equally in all age groups. The incidence is higher in the elderly where estimates are as high as fifty percent experience nocturnal cramps on multiple occasions.

In another embodiment, the composition of the presently disclosed and claimed inventive concept(s) one or more counterirritants, including but not limited to, menthol, methyl salicylate, camphor, and trolamine salicylate, may be added to the composition in combination with the effective amount of the creatine compound and the at least one essential oil.

Generally, the compositions of the presently claimed and disclosed inventive concept(s) are administered in a pharmaceutically or a therapeutically effective dose which is an amount of the composition which when topically applied provides relief from cramping or recurrence. The amount of the compound actually administered in a therapeutic setting may, typically be determined by an athletic trainer or a physician, in the light of the relevant circumstances.

The compositions used in the methods of the presently disclosed and claimed inventive concept(s) for preventing and/or treating exercise-induced muscle cramps, stiffness, pain or spasms, are administered to a subject at therapeutically effective doses. A therapeutically effective dose refers to that amount of the creatine compound sufficient to result in the amelioration of symptoms of muscle cramps, muscle stiffness, muscle pain, or muscle spasms, or recurrence thereof.

The compositions of the presently claimed and disclosed inventive concept(s) are to be administered topically which may include transdermal absorption (or via other epithelial administration, where desired). In such compositions, the creatine compound comprises from about 0.1% to about 90% of the weight of the composition for example, the creatine compound may comprise, for example, at least 0.1%, or 0.25%, or 0.5%, or 0.75%, or 1%, or 2%, or 3%, or 4%, or 5%, or 6%, or 7%, or 8%, or 9%, or 10%, or 12.5%, or 15%, or 17.5%, or 20%, or 25%, or 30%, or 35%, or 40%, or 50%, or 60%, or 70%, or 80%, or 90%, by weight of the composition, which optionally comprises at least one essential oil. The one or more essential oils may comprise, in one non-limiting embodiment, from about 0.01% to about 10% by weight, for example, of the composition of the creatine compound. The essential oil may comprise, for example, from about 0.1% to about 7.5% of the composition. The essential oil may comprise from about 1% to about 5%, for example, of the composition. In other embodiments the one or more essential oils may comprise up to, but is not limited to, about 0.01%, or 0.05%, or 0.1%, or 0.5%, or 1.0%, or 2%, or 3%, or 4%, or 5%, or 6%, or 7%, or 8%, or 9%, or 10% by weight of the composition.

Topical cosmetic forms and topical pharmaceutical dosing forms can include lotions, gels, creams, ointments, sprays, suspensions, pastes, and “sticks.” Lotions commonly employ a water or alcohol base. Gels are semi-solid emulsions or suspensions. Creams generally contain a significant proportion of water in their base while ointments are commonly more oily.

Liquid forms of the composition, such as lotions, may include a suitable aqueous or non-aqueous vehicle with buffers, suspending and dispensing agents, thickeners, penetration enhancers, and the like. More solid forms such as creams or pastes or the like may include, for example, any of the following ingredients: water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

The above-described components for liquid, semisolid and solid (e.g., “stick”) compositions are merely representative. Other materials as well as processing techniques and the like are set forth in Remington: The Science and Practice of Pharmacy, 21^{st} ed., which is hereby expressly incorporated herein by reference in its entirety.

In one embodiment, when the creatine composition is to be administered transdermally, the composition may be a liquid solution or gels. In these settings the concentration of creatine is generally from about 0.1% to about 25% by weight, and more particularly from 1% to 10%, of the composition, with the remainder being aqueous mixed or nonaqueous vehicle, such as alcohols and the like, suspending agents, gelling agents, surfactant, and the like, and optionally with at least one essential oil as described elsewhere herein. Examples of suitable such materials are described below.

The compositions can also be administered in sustained release transdermal forms or from transdermal sustained release drug delivery systems. A description of representative sustained release materials can be found in the incorporated materials in Remington: The Science and Practice of Pharmacy, 21^{st} ed.

The creatine compounds and the one or more optional essential oils of the compositions are combined with a pharmaceutically acceptable carrier. Such a composition may contain, in addition to the creatine compound, essential oil (when present) and carrier, one or more diluents, fillers, salts, buffers, stabilizers, solubilizers, and other materials well known in the art. Suitable carriers, vehicles and other components of the formulation are described, for example, in Remington: The Science and Practice of Pharmacy, 21^{st} ed. The term “pharmaceutically acceptable,” as noted above, means a non-toxic material that does not interfere with the effectiveness of the biological activity of the active ingredient(s).

The creatine compositions of the presently disclosed and claimed inventive concept(s) may be in the form of liposomes in which the creatine compound, and/or essential oil, is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

An effective amount of the composition of the presently disclosed and claimed inventive concept(s) refers to an amount which is effective in controlling or reducing a muscle cramp, muscle cramp, muscle spasm, muscle pain, or muscle stiffness, and/or effective in inhibiting the short term reoccurrence thereof. The term “controlling” is intended to refer to all processes wherein there may be a slowing, interrupting, arresting, or stopping, or reoccurrence of the muscle cramp, muscle pain, muscle stiffness, muscle spasm, and does not necessarily indicate a total elimination of the muscle cramping, muscle spasm, muscle pain, or muscle stiffness.

The actual dose will vary with the patient's overall condition, the seriousness of the symptoms, and counter indications. As used herein, the term “therapeutically effective amount” also means the total amount of each active component of the pharmaceutical composition or method that is sufficient to show a meaningful patient benefit, i.e., reduction of muscle cramp, muscle spasm, muscle pain, or muscle stiffness, and/or inhibition of the reoccurrence thereof. When applied to an individual active ingredient (e.g., the creatine compound and/or the essential oil), administered alone, the term refers to that ingredient alone. When applied to a combination, the term refers to combined amounts of the active ingredients (e.g., creatine compound and essential oil) that result in the therapeutic effect, whether administered in combination, serially or simultaneously.

The pharmaceutical composition of the presently disclosed and claimed inventive concept(s) may also contain stabilizers, preservatives, buffers, antioxidants, or other additive known to those of skill in the art.

The compositions of the presently disclosed and claimed inventive concept(s) can be manufactured utilizing techniques known in the art. Typically the therapeutically effective amount of the creatine compound (and optionally an essential oil) will be admixed with a pharmaceutically acceptable carrier.

One method useful in controlling the duration of action of the creatine compound by controlled release preparations is to incorporate the creatine compound into particles of a polymeric material such as polyesters, polyamides, polyamino acids, hydrogels, poly(lactic acid), ethylene vinylacetate copolymers, copolymer micelles of, for example, PEG and poly(1-aspartamide). It is also possible to entrap the creatine compounds in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

Examples are provided hereinbelow. However, the presently disclosed and claimed inventive concept(s) is to be understood to not be limited in its applicant to the specific experimentation, results and laboratory procedures. Rather, the Examples are simply provided as one of various embodiments and are meant to be exemplary, not exhaustive.

### EXAMPLES

### Formulation Example 1

TABLE 5
Formulation 1 composition

| Ingredient | Amount | |
|---|---|---|
| Creatine monohydrate¹ | 256 | g |
| Chamomile Oil² | 22.4 | ml |
| Lavender Oil² | 22.4 | ml |
| Eucalyptus Oil² | 12.5 | ml |
| Clary Sage Oil² | 6.8 | ml |
| Lotion Base³ (carrier) | 3785 | ml |

¹Source: AlzChem AG, Trostberg, Germany
²Source: Now Foods, Bloomingdale, IL. 60108
³Source: Bulk Apothecary, Streetboro, Ohio 44241

### Experimental Example 1

An open label study using the composition of Formulation Example 1 was conducted at seven athletic programs over a 30 day period in September-October, 2012, by athletic trainers under the supervision of an orthopedic surgeon. Individuals suffering from muscle cramps were treated by topical application of the composition to the affected limb or area. Each applied dose was sufficient to provide approximately 0.5 to 2.5 g of the creatine compound to the affected limb or area. The primary endpoint was cessation of exercise-related muscular cramps, with a secondary endpoint of non-recurrence for remainder of sport event or practice period. All facilities provide fluid and electrolyte replacement ad lib for the athletes before and during activities. The amount of the creatine compound in the dose is approximately 0.01 g to about 3 g.

Prior to initiation of the experimental protocol participating athletic trainers were asked to evaluate current practices for treating exercise-related muscular cramps and based upon past experiences the effectiveness of the existing technology. Typical responses characterized the state of the art currently and range from “the most effective treatment is massage and stretching” and “once an exercise related muscle cramp begins even with massage, electrolytes and stretching to stop the cramp, the muscle cramp frequently reoccurs repeated when the athlete resumes exercise”. “None of the conventionally available cramp relief topical products appear to work any better than plain massage”. This limited utility of existing modalities was clearly observed during a nationally televised NCAA division I football game on Sep. 1, 2012. During the game, both teams experienced multiple exercise related muscular cramping events and reoccurrences. After multiple injury related timeouts, the sideline training area was observed to be using kitchen mustard and massage as the treatment of choice.

TABLE 6
Results of Experiment using Formulation 1.
Results

| | Athletic Trainer | | | |
|---|---|---|---|---|
| Factor | “A” | “B” | “C” | “D” |
| Athletic programs served by Athletic Trainer | 4 | 1 | 1 | 1 |
| Number of players who experienced muscle | 10 | 3 | 30 | 3 |
| cramping | | | | |
| Total number of times composition was | 20 | 8 | 30 | 3 |
| applied | | | | |
| Total times applied during cramping | 20 | 6 | 25 | 3 |
| Total times applied prophylactically (before | 0 | 2 | 5 | 0 |
| initiation of exercise activity) | | | | |
| Total times player had reoccurrence of | 0 | 0 | 0 | 0 |
| cramping | | | | |
| Percentage effectiveness when applied after | 100% | 100% | 96% | 100% |
| cramping | | | | |
| Percentage effectiveness in preventing | N/A | 100% | 80% | N/A |
| cramping when applied prophylactically | | | | |

The results shown in Table 6 clearly demonstrate the effectiveness of a composition comprising a creatine compound and at least one essential oil in the treatment and cessation of episodes of muscle cramps, and in the inhibition of a reoccurrence of the muscle cramp and in the prevention of muscle cramping by prophylactic treatment with the composition. Treatment of an episode of muscle cramping is generally effective within 3 to 10 minutes of topical application of the creatine composition or within 3 to 15 minutes to 3 to 20 minutes. Inhibition of reoccurrence of the muscle cramp after the initial event (episode) is effective for up to at least one of 1, 2, 3, 4, 5, 6, 7, 8, 9, 10, 11, 12, 13, 14, 15, 16, 17, 18, 19, 20, 21, 22, 23, or 24 hours.

The presently disclosed and claimed inventive concept(s) are not to be limited in scope by the specific formulations and embodiments described herein, since such formulations and embodiments are intended only as individual illustrations of certain aspects of the presently disclosed and claimed inventive concept(s), and any functionally equivalent embodiments are within the scope of the presently disclosed and claimed inventive concept(s). Indeed, various modifications of the methods of the presently disclosed and claimed inventive concepts, in addition to those shown and described herein, will become apparent to those skilled in the art from the foregoing description.

Further, although the presently disclosed and claimed inventive concept(s) and the advantages thereof have been described in detail, it should be understood that various changes, substitutions and alterations can be made herein without departing from the spirit and scope of the presently disclosed and claimed inventive concept(s) as defined by the appended claims. Moreover, the scope of the present application is not intended to be limited to the particular embodiments of the process, items of manufacture, compositions of matter, means, methods and steps described in the specification. As one of ordinary skill in the art will readily appreciate from the disclosure of the presently disclosed and claimed inventive concept(s), processes, items of manufacture, compositions of matter, means, methods, or steps, presently existing or later to be developed that perform substantially the same function or achieve substantially the same result as the corresponding embodiments described herein may be utilized according to the presently disclosed and claimed inventive concept(s). Accordingly, the appended claims are intended to include within their scope such processes, items of manufacture, compositions of matter, means, methods, or steps.

1. A method for treating and/or inhibiting recurrence of at least one of muscle cramps, muscle spasms, muscle stiffness associated with muscle cramps and/or spasms, and muscle pain associated with muscle cramps and/or spasms in a mammalian subject in need of such treatment, wherein the muscle cramps and/or spasms are not exercise-associated, comprising:
topically administering to the subject a composition comprising (1) an effective amount of a creatine compound, and (2) a pharmaceutically-acceptable carrier, thereby treating and/or inhibiting recurrence of at least one of a non-exercise-associated muscle cramp, non-exercise-associated muscle stiffness, non-exercise-associated muscle pain, and non-exercise-associated muscle spasm in the subject.

2. The method of claim 1, wherein the composition comprises from about 0.1% to about 90% by weight of the creatine compound.

3. The method of claim 1, wherein the composition comprises from about 1% to 50% by weight of the creatine compound.

4. The method of claim 1, wherein the composition comprises from about 2% to about 10% by weight of the creatine compound.

5. The method of claim 1, wherein the composition further comprises an effective amount of at least one essential oil.

6. The method of claim 5, wherein the at least one essential oil comprises from about 0.01% to about 10% by weight of the composition.

7. The method of claim 5, wherein the at least one essential oil comprises from about 0.1% to about 1.0% by weight of the composition.

8. The method of claim 5, wherein the at least one essential oil is selected from the group consisting of sesame oil, macadamia nut oil, tea tree oil, evening primrose oil, Spanish sage oil, Spanish rosemary oil, coriander oil, cardamom oil, camphor oil, thyme oil, pimento berries oil, rose oil, anise oil, almond oil, balsam oil, bergamot oil, rosewood oil, cedar oil, cedar leaf oil, cinnamon oil, chamomile oil, sage oil, clary sage oil, clove oil, clove leaf oil, cypress oil, eucalyptus oil, fennel oil, sea fennel oil, fir tree oil, frankincense oil, geranium oil, ginger oil, grapefruit oil, jasmine oil, jojoba oil, juniper oil, lavender oil, lemon oil, lemongrass oil, lime oil, mandarin oil, marjoram oil, menthol oil, myrrh oil, neroli oil, orange oil, patchouli oil, pepper oil, black pepper oil, petitgrain oil, pine seed oil, pine needle oil, rose otto oil, rosemary oil, sandalwood oil, spearmint oil, spikenard oil, vetiver oil, white pine oil, wintergreen oil, and ylang ylang.

9. The method of claim 1, wherein the creatine compound of the composition is creatine monohydrate and the composition further comprises at least one of chamomile oil, eucalyptus oil, clary sage oil, and lavender oil.

10. The method of claim 1, wherein the composition is formulated into a gel, a cream, an ointment, a paste, a lotion, a spray, a suspension, or a stick.

11. The method of claim 1, wherein the subject receives relief from the muscle cramp, muscle stiffness, muscle pain, and/or muscle spasm within 3-20 minutes of topical application of the composition.

12. The method of claim 1, wherein recurrence of the muscle cramp is inhibited for up to 24 hours after an initial muscle cramp episode.

13. A method for treating and/or inhibiting recurrence of at least one of muscle cramps, muscle spasms, muscle stiffness associated with muscle cramps and/or spasms, and muscle pain associated with muscle cramps and/or spasms in a mammalian subject in need of such treatment, wherein the muscle cramps and/or spasms are not exercise-associated, comprising:
topically administering to the subject a composition comprising (1) an effective amount of a creatine compound, (2) an effective amount of at least one essential oil, and (3) a pharmaceutically-acceptable carrier, thereby treating and/or inhibiting recurrence of at least one of a non-exercise-associated muscle cramp, non-exercise-associated muscle stiffness, non-exercise-associated muscle pain, and non-exercise-associated muscle spasm in the subject.

14. The method of claim 13, wherein the composition comprises from about 0.1% to about 90% by weight of the creatine compound.

15. The method of claim 13, wherein the composition comprises from about 2% to about 10% by weight of the creatine compound.

16. The method of claim 13, wherein the at least one essential oil comprises from about 0.01% to about 10% by weight of the composition.

17. The method of claim 13, wherein the at least one essential oil comprises from about 0.1% to about 1.0% by weight of the composition.

18. The method of claim 13, wherein the at least one essential oil is selected from the group consisting of sesame oil, macadamia nut oil, tea tree oil, evening primrose oil, Spanish sage oil, Spanish rosemary oil, coriander oil, cardamom oil, camphor oil, thyme oil, pimento berries oil, rose oil, anise oil, almond oil, balsam oil, bergamot oil, rosewood oil, cedar oil, cedar leaf oil, cinnamon oil, chamomile oil, sage oil, clary sage oil, clove oil, clove leaf oil, cypress oil, eucalyptus oil, fennel oil, sea fennel oil, fir tree oil, frankincense oil, geranium oil, ginger oil, grapefruit oil, jasmine oil, jojoba oil, juniper oil, lavender oil, lemon oil, lemongrass oil, lime oil, mandarin oil, marjoram oil, menthol oil, myrrh oil, neroli oil, orange oil, patchouli oil, pepper oil, black pepper oil, petitgrain oil, pine seed oil, pine needle oil, rose otto oil, rosemary oil, sandalwood oil, spearmint oil, spikenard oil, vetiver oil, white pine oil, wintergreen oil, and ylang ylang.

19. The method of claim 13, wherein the composition is formulated into a gel, a cream, an ointment, a paste, a lotion, a spray, a suspension, or a stick.

20. The method of claim 13, wherein the subject receives relief from the muscle cramp, muscle stiffness, muscle pain, and/or muscle spasm within 3-20 minutes of topical application of the composition.

21. The method of claim 13, wherein recurrence of the muscle cramp is inhibited for up to 24 hours after an initial muscle cramp episode.

22. The method of claim 1, wherein the non-exercise-associated muscle cramps and/or spasms are selected from the group consisting of nocturnal cramps/spasms, heat cramps/spasms, writer's cramps/spasms, premenstrual cramps/spasms, and age-related cramps/spasms.

23. The method of claim 13, wherein the non-exercise-associated muscle cramps and/or spasms are selected from the group consisting of nocturnal cramps/spasms, heat cramps/spasms, writer's cramps/spasms, premenstrual cramps/spasms, and age-related cramps/spasms.
